(19) United States
(12) Patent Application Publication
(10) Pub. No.: US 2017/0112980 A1
(43) Pub. Date: Apr. 27, 2017

(54) Mucus Suction Assembly

(71) Applicant: Russell Purnell, Jasper, TN (US)

(72) Inventor: Russell Purnell, Jasper, TN (US)

(21) Appl. No.: 14/923,510

(22) Filed: Oct. 27, 2015

(51) Int. Cl. A61M 1/00

(52) U.S. Cl. CPC A61M 1/0066; A61M 1/0001; A61M 2210/0618

(57) ABSTRACT

A mucus suction assembly for suctionally removing mucus from a nasal cavity includes a suction unit that generates a suction pressure. A mucus remover is removably coupled to the suction unit such that the mucus remover communicates the suction pressure. The mucus remover may be inserted into a nasal cavity thereby facilitating the mucus remover to suctionally remove mucus from the nasal cavity.

### BACKGROUND OF THE DISCLOSURE

### Field of the Disclosure

The disclosure relates to suction devices and more particularly pertains to a new suction device for suctionally removing mucus from a nasal cavity.

### SUMMARY OF THE DISCLOSURE

An embodiment of the disclosure meets the needs presented above by generally comprising a suction unit that generates a suction pressure. A mucus remover is removably coupled to the suction unit such that the mucus remover communicates the suction pressure. The mucus remover may be inserted into a nasal cavity thereby facilitating the mucus remover to suctionally remove mucus from the nasal cavity.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top perspective view of a mucus suction assembly according to an embodiment of the disclosure.

FIG. 2 is a front view of an embodiment of the disclosure.

FIG. 3 is a back view of an embodiment of the disclosure.

FIG. 4 is a front cut-away view of an embodiment of the disclosure.

FIG. 5 is a perspective in-use view of an embodiment of the disclosure.

### DESCRIPTION OF THE PREFERRED EMBODIMENT

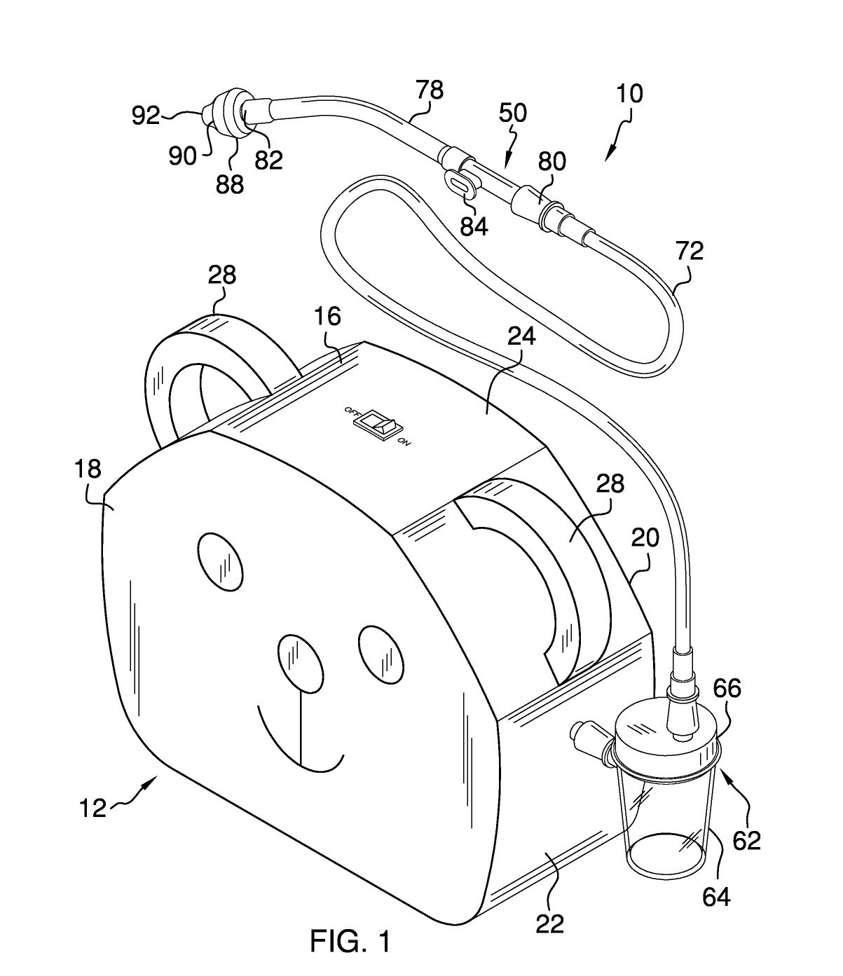

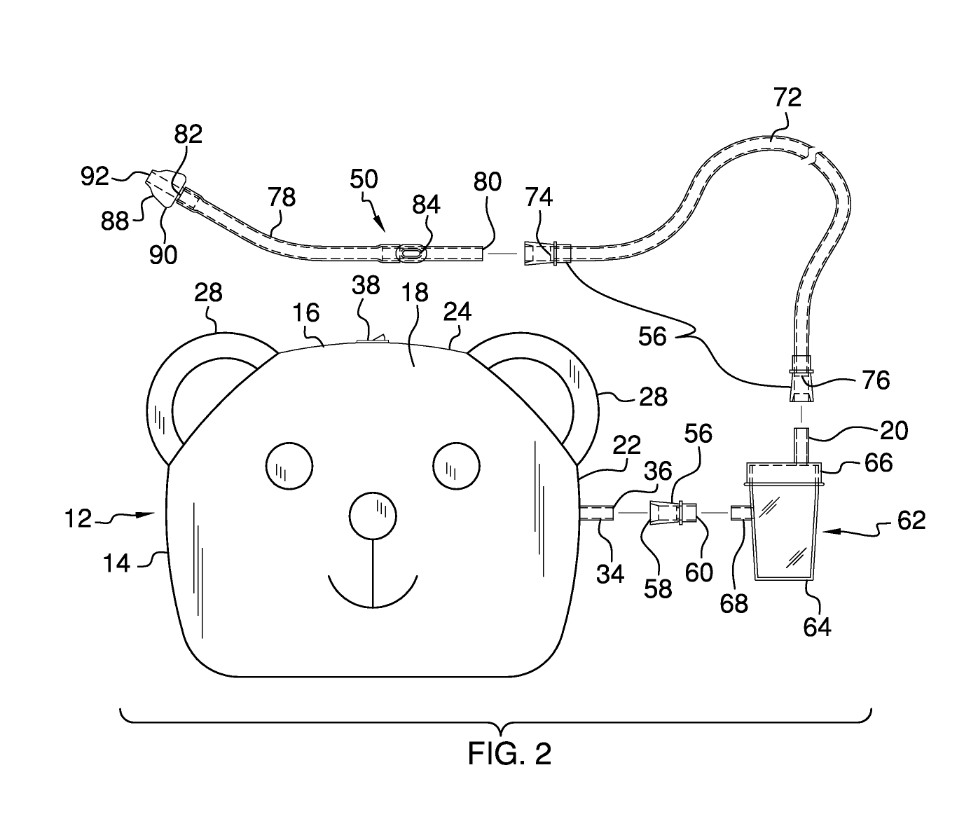

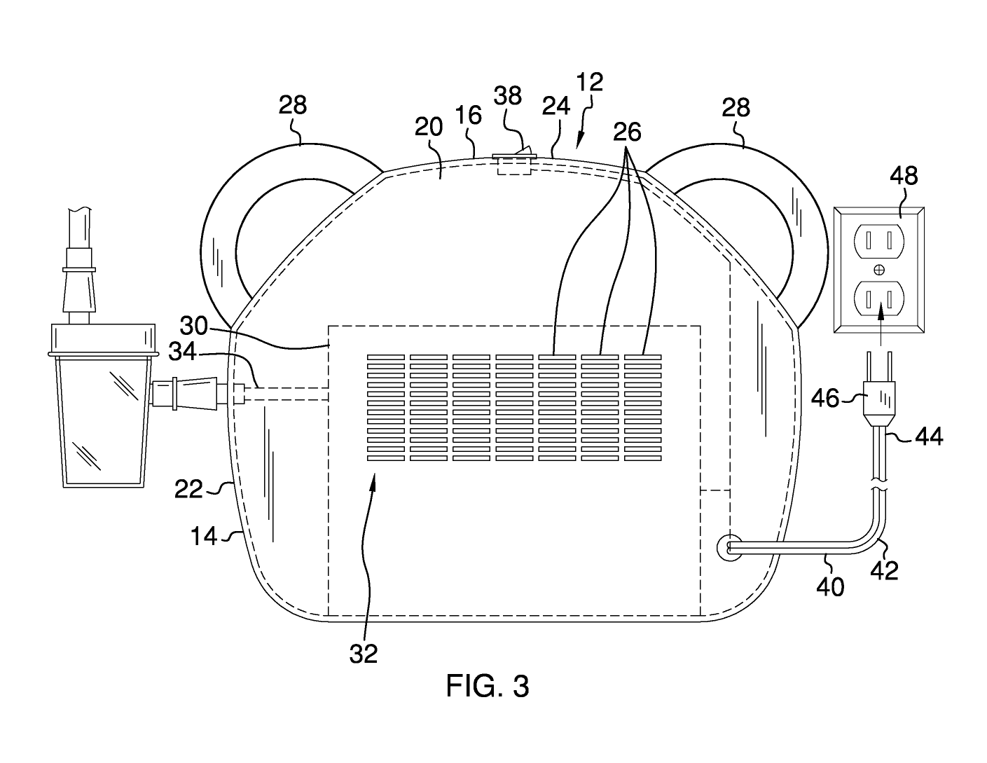

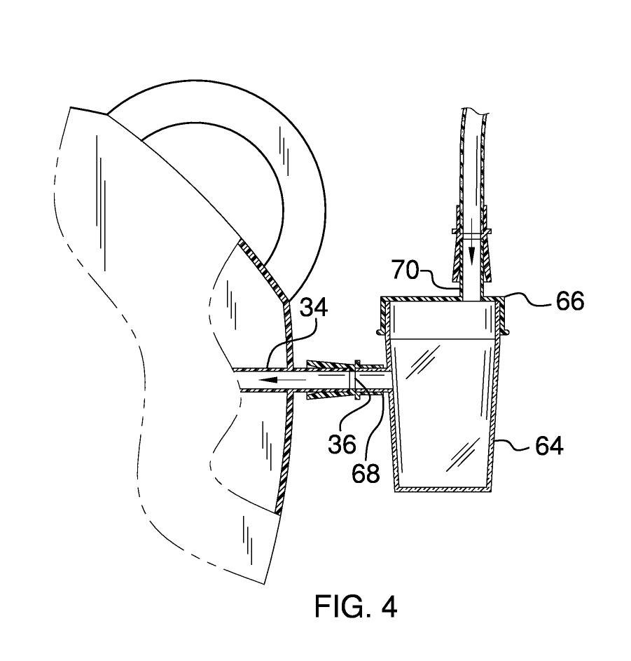

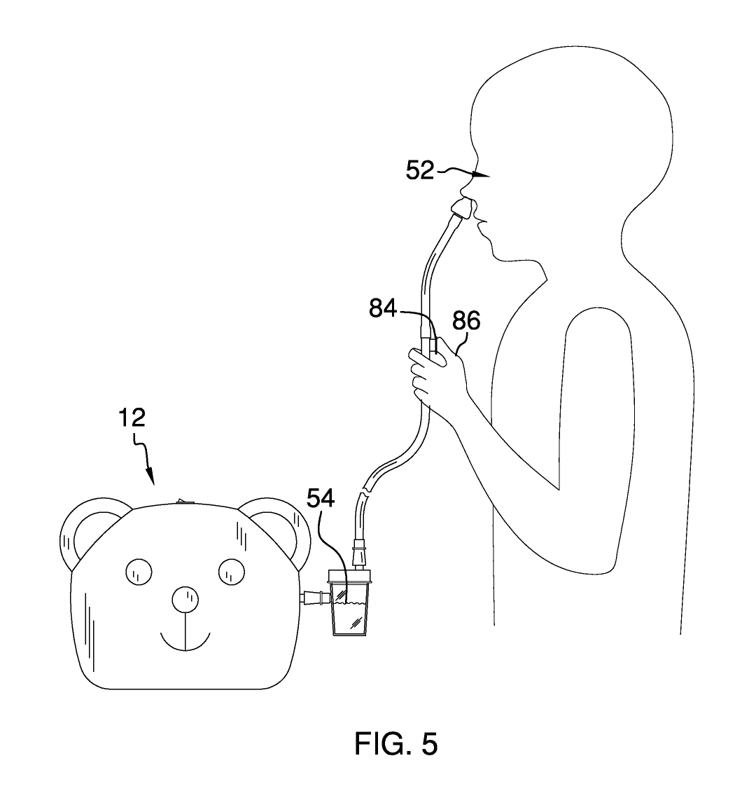

With reference now to the drawings, and in particular to FIGS. 1 through 5 thereof, a new suction device embodying the principles and concepts of an embodiment of the disclosure and generally designated by the reference numeral 10 will be described.

As best illustrated in FIGS. 1 through 5, the mucus suction assembly 10 generally comprises a suction unit 12 that may generate a suction pressure. The suction unit 12 comprises a housing 14 that has an outer wall 16, a front wall 18 and a back wall 20. The outer wall 16 has a first lateral side 22 and a top side 24. The back wall 20 has a plurality of openings 26 extending therethrough. The top side 24 may be shaped to form a head of an animal such as a bear or the like. The front wall 18 may have an image of a face printed thereon to compliment the animal head. A pair of handles 28 may be provided and each of the handles 28 may be coupled to the top side 24. Each of the handles 28 may be curved such that each of the handles 28 forms ears of the animal.

A vacuum pump 30 is positioned within the housing 14 and the vacuum pump 30 has an intake 32 and an outlet 34. The intake 32 is in fluid communication with the openings 26. The outlet 34 extends outwardly through the first lateral side 22. The outlet 34 has a distal end 36 with respect to the first lateral side 22 and the distal end 36 is open. The vacuum pump 30 may be an electrical vacuum pump or the like.

A switch 38 is positioned on the top side 24 such that the switch 38 may be manipulated. The switch 38 is electrically coupled to the vacuum pump 30. A power supply 40 is positioned within the housing 14 and the power supply 40 is electrically coupled to the switch 38. The power supply 40 includes a cord 42 extending outwardly from the back wall 20. The cord 42 has a distal end 44 with respect to the back wall 20 and the distal end 44 of the cord 42. A plug 46 is electrically coupled to the distal end 44 of the cord 42. The plug 46 may be electrically coupled to a power source 48 and the power source 48 may comprise an electrical outlet or the like.

A mucus remover 50 is removably coupled to the suction unit 12 such that the mucus remover 50 may communicate the suction pressure. The mucus remover 50 may be inserted into a nasal cavity 52 thereby facilitating the mucus remover 50 to suctionally remove mucus 54 from the nasal cavity 52. The mucus remover 50 comprises a plurality of couplers 56 and each of the couplers 56 has a first end 58 and a second end 60. Each of the first end 58 and the second end 60 of each of the couplers 56 is open and each of the couplers 56 is substantially hollow.

A cup 62 is provided that has a body 64 and a lid 66. The lid 66 is removably coupled to the body 64. The body 64 has an outlet 68 extending outwardly therefrom and the lid 66 has an inlet 70 extending upwardly therefrom. One of the couplers 56 is fluidly coupled between the outlet 68 of the body 64 and the intake 32 of the vacuum pump 30. Thus, the cup 62 is in fluid communication with the vacuum pump 30.

A hose 72 is provided that has a first end 74 and a second end 76. One of the couplers 56 is fluidly coupled between the first end 74 and the inlet 70. Thus, the hose 72 is in fluid communication with the vacuum pump 30. The hose 72 may be comprised of a flexible material such as surgical hose or the like.

A nozzle 78 is provided that has a primary end 80, a secondary end 82 and a valve 84. Each of the primary end 80, the secondary end 82 and the valve 84 is open. One of the couplers 56 is fluidly coupled between the secondary end 82 and the second end 76 of the hose 72. Thus, the nozzle 78 is in fluid communication with the vacuum pump 30. The valve 84 is positioned between the primary end 80 and the secondary end 82 such that the valve 84 may have a finger 86 selectively placed thereon. Thus, the valve 84 selectively inhibits and permits the secondary end 82 to communicate the suction.

A tip 88 is fluidly coupled to the secondary end 82. The tip 88 has an outer wall 90 and a distal end 92 with respect to the secondary end 82 and the distal end 92 of the tip 88 is open. The outer wall flares 90 outwardly between the distal end 82 of the tip 88 and the secondary end 82. The tip 88 may be inserted into the nasal cavity 52 thereby facilitating the tip 88 to suctionally remove the mucus 54 from the nasal cavity 52. The outer wall 90 of the tip 88 engages the nasal cavity 52 such that the tip 88 forms a fluid impermeable seal with the nasal cavity 52.

In use, the nozzle 78, the hose 72 and the cup 62 are all fluidly coupled to the vacuum pump 30. The switch 38 is manipulated to turn the vacuum pump 30 on. The nozzle 78 is manipulated to insert the tip 88 into the nasal cavity 52. The valve 84 is covered with the finger 86 thereby facilitating the mucus 54 to be suctionally removed from the nasal cavity 52. The mucus 54 is deposited in the body 64 of the cup 62. The lid 66 is removed from the body 64 and the body 64 is emptied when the cup 62 becomes filled with mucus 54.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A mucus suction assembly comprising:
a suction unit being configured to generate a suction pressure; and
a mucus remover being removably coupled to said suction unit wherein said mucus remover is configured to communicate the suction pressure, said mucus remover being configured to be inserted into a nasal cavity thereby facilitating said mucus remover to suctionally remove mucus from the nasal cavity.

2. The assembly according to claim 1, wherein said suction unit comprises a housing having an outer wall, a back wall and a front wall, said outer wall having a first lateral side and a top side, said back wall having a plurality of openings extending therethrough.

3. The assembly according to claim 2, further comprising a vacuum pump being positioned within said housing, said vacuum pump having an intake and an outlet, said intake being in fluid communication with said openings, said outlet extending outwardly through said first lateral side, said outlet having a distal end with respect to said first lateral side, said distal end being open.

4. The assembly according to claim 3, further comprising a switch being positioned on said top side wherein said switch is configured to be manipulated, said switch being electrically coupled to said vacuum pump.

5. The assembly according to claim 4, further comprising a power supply being positioned within said housing, said power supply being electrically coupled to said switch, said power supply including a cord extending outwardly from said back wall, said cord having a distal end with respect to said back wall, said distal end of said cord having a plug being electrically coupled thereto wherein said plug is configured to be electrically coupled to a power source.

6. The assembly according to claim 1, wherein said mucus remover comprises a plurality of couplers, each of said couplers having a first end and a second end, each of said first end and said second end of each of said couplers being open, each of said couplers being substantially hollow.

7. The assembly according to claim 6, further comprising:
a vacuum pump; and
a cup having a body and a lid, said lid being removably coupled to said body, said body having an outlet extending outwardly therefrom, said lid having an inlet extending upwardly therefrom, one of said couplers being fluidly coupled between said outlet of said body and said intake of said vacuum pump such that said cup is in fluid communication with said vacuum pump.

8. The assembly according to claim 7, further comprising a hose having a first end and a second end, one of said couplers being fluidly coupled between said first end and said inlet such that said hose is in fluid communication with said vacuum pump.

9. The assembly according to claim 8, further comprising a nozzle having a primary end, a secondary end and a valve, each of said primary end, said secondary end and said valve being open, one of said couplers being fluidly coupled between said secondary end and said second end of said hose wherein said nozzle is in fluid communication with said vacuum pump, said valve being positioned between said primary end and said secondary end wherein said valve is configured to have a finger selectively placed thereon thereby facilitating said valve to inhibit and permit said secondary end to communicate the suction.

10. The assembly according to claim 9, further comprising a tip being fluidly coupled to said secondary end, said tip having an outer wall and a distal end with respect to said secondary end, said distal end of said tip being open, said outer wall flaring outwardly between said distal end of said tip and said secondary end, said tip being configured to be inserted into the nasal cavity thereby facilitating said tip to suctionally remove the mucus from the nasal cavity.

11. A mucus suction assembly comprising:
a suction unit being configured to generate a suction pressure, said suction unit comprising:
a housing having an outer wall, a back wall and a front wall, said outer wall having a first lateral side and a top side, said back wall having a plurality of openings extending therethrough,
a vacuum pump being positioned within said housing, said vacuum pump having an intake and an outlet, said intake being in fluid communication with said openings, said outlet extending outwardly through said first lateral side, said outlet having a distal end with respect to said first lateral side, said distal end being open,
a switch being positioned on said top side wherein said switch is configured to be manipulated, said switch being electrically coupled to said vacuum pump, and
a power supply being positioned within said housing, said power supply being electrically coupled to said switch, said power supply including a cord extending outwardly from said back wall, said cord having a distal end with respect to said back wall, said distal end of said cord having a plug being electrically coupled thereto wherein said plug is configured to be electrically coupled to a power source;
a mucus remover being removably coupled to said suction unit wherein said mucus remover is configured to communicate the suction pressure, said mucus remover being configured to be inserted into a nasal cavity thereby facilitating said mucus remover to suctionally remove mucus from the nasal cavity, said mucus remover comprising:
a plurality of couplers, each of said couplers having a first end and a second end, each of said first end and said second end of each of said couplers being open, each of said couplers being substantially hollow,
a cup having a body and a lid, said lid being removably coupled to said body, said body having an outlet extending outwardly therefrom, said lid having an inlet extending upwardly therefrom, one of said couplers being fluidly coupled between said outlet of said body and said intake of said vacuum pump such that said cup is in fluid communication with said vacuum pump,
a hose having a first end and a second end, one of said couplers being fluidly coupled between said first end and said inlet such that said hose is in fluid communication with said vacuum pump,
a nozzle having a primary end, a secondary end and a valve, each of said primary end, said secondary end and said valve being open, one of said couplers being fluidly coupled between said secondary end and said second end of said hose wherein said nozzle is in fluid communication with said vacuum pump, said valve being positioned between said primary end and said secondary end wherein said valve is configured to have a finger selectively placed thereon thereby facilitating said valve to inhibit and permit said secondary end to communicate the suction, and
a tip being fluidly coupled to said secondary end, said tip having an outer wall and a distal end with respect to said secondary end, said distal end of said tip being open, said outer wall flaring outwardly between said distal end of said tip and said secondary end, said tip being configured to be inserted into the nasal cavity thereby facilitating said tip to suctionally remove the mucus from the nasal cavity.
